(19) United States
(12) Patent Application Publication
(10) Pub. No.: US 2007/0196892 A1
(43) Pub. Date: Aug. 23, 2007

(54) METHOD OF CONVERTING A FERMENTATION BYPRODUCT INTO OXYGEN AND BIOMASS AND RELATED SYSTEMS

(76) Inventors: David J. Winsness, Alpharetta, GA (US); Richard Krablin, Stewartsville, NJ (US); Kevin E. Kreisler, Mt. Arlington, NJ (US)

Correspondence Address: KING & SCHICKLI, PLLC, 247 NORTH BROADWAY, LEXINGTON, KY, 40507, US

(21) Appl. No.: 11/688,502

(22) Filed: Mar. 20, 2007

Related U.S. Application Data
(63) Continuation of application No. PCT/US07/62551, filed on Feb. 22, 2007.
(60) Provisional application No. 60/842,398, filed on Sep. 5, 2006.
(60) Provisional application No. 60/775,663, filed on Feb. 22, 2006.

(51) Int. Cl. C12P 1/00; C12P 7/06

(52) U.S. Cl. 435/41; 435/161

(57) ABSTRACT

In one aspect, the invention relates to a method of converting byproducts of a fermentation process into oxygen and biomass. Related methods, systems, and other aspects are also described.

This application is a continuation of PCT/US07/62551, filed Feb. 22, 2007, and claims the benefit of U.S. Provisional Patent Application Ser. No. 60/775,663, filed Feb. 22, 2006, and U.S. Provisional Patent Application Ser. No. 60/842,398, filed Sep. 5, 2006, the disclosures of all of which applications are incorporated herein by reference.

### COPYRIGHT STATEMENT

A portion of the disclosure of this document contains material subject to copyright protection. No objection is made to the facsimile reproduction of the patent document or this disclosure as it appears in the Patent and Trademark Office files or records, but any and all rights in the copyright(s) are otherwise reserved.

### TECHNICAL FIELD

The present invention relates generally to fermentation processes and, more particularly, to a method of converting a byproduct from a fermentation process into oxygen and biomass.

### BACKGROUND OF THE INVENTION

Over the past thirty years, significant attention has been given to the production of ethyl alcohol, or “ethanol,” for use as an alternative fuel. Ethanol not only burns cleaner than fossil fuels, but also can be produced using corn, a renewable resource. At present, “dry milling” plants in the United States alone produce billions of gallons of ethanol per year. Additional plants presently under construction are expected to add hundreds of millions gallons to this total in an effort to meet the current high demand. Further gaining widespread attention is a competing renewable fuel that may be made from oil (including that recovered from the ethanol production process) known generally as “biodiesel.”

As noted in the foregoing discussion, a popular method of producing ethanol from corn is known as “dry milling.” As is well known in the industry, the dry milling process utilizes the starch in the corn to produce the ethanol through fermentation. Besides creating a waste stream comprised of byproducts termed “whole stillage” (which may be further separated into products commonly referred to as “distillers wet grains” and “thin stillage”), the process also produces waste in the form of carbon dioxide gas, or CO₂. The same is true of an alternative process for ethanol production called “wet milling,” the main difference from dry milling being that the corn is soaked beforehand.

Unfortunately, CO₂ reflects infrared radiation. Consequently, when released into the atmosphere in excessive amounts, it retains heat and makes the surface temperature warmer. This is deleterious for obvious reasons. At present growth rates, estimated CO₂ levels in the atmosphere will increase from 350 ppmv (at present) to 750 ppmv in as little as 80 years. Indeed, leveling CO₂ concentrations at 550 ppmv requires reducing net CO₂ emissions by over 60% from 1990 levels during the next 100 years.

A prior proposal for a possible partial solution to the foregoing problem involves using biological agents to feed on the CO₂-laden flue gas resulting from the combustion of non-renewable fossil fuels. Specifically, U.S. Pat. No. 6,667,171 to Bayless et al. (the disclosure of which is incorporated herein by reference) describes one type of system for passing flue gas including CO₂ over a plurality of porous membranes supporting a colony of microbial agents, such as cyanobacteria. These bacteria thrive on the CO₂ and, in the process, convert it to harmless oxygen and create a significant amount of starchy biomass. The oxygen can simply be released to the environment, while the biomass harvested and used to produce products, such as ethanol or biodiesel.

However, harvesting using this type of arrangement normally takes place at a location far removed from where ethanol or biodiesel production occurs. Thus, as acknowledged by Bayless et al., inefficient post-harvesting transport of the biomass over long distances is potentially required. Not only does this reduce lack efficiency, but is also tends to contribute further to the problem sought to be resolved, since ethanol and biodiesel production using the harvested biomass results in the generation of additional CO₂.

Accordingly, a need exists for a more efficient and economical manner of converting byproducts from fermentation into useable, environmentally safe products, such as oxygen and biomass, and without releasing significant amounts of CO₂ into the atmosphere.

### SUMMARY OF THE INVENTION

In accordance with a first aspect of the invention, a method of converting byproducts into oxygen and biomass is disclosed. The method comprises fermenting corn to produce ethanol and a gaseous byproduct, recovering the gaseous byproduct, and using the gaseous byproduct to generate the oxygen and biomass.

In one embodiment, the gaseous byproduct consists substantially of CO₂. The method may also include the step of using the biomass created to produce the gaseous byproduct. Alternatively or additionally, the method may further include the step of using the biomass created to produce ethanol (such as by fermenting the biomass). Still another option is to extract oil from the biomass.

The method may further include the step of dry or wet milling corn prior to the fermenting step. Preferably, the dry milling step includes cooking milled corn using a boiler. In that case, the method further includes using the boiler exhaust to generate the biomass.

The method may still further involve the step of using the gaseous byproduct to generate the biomass includes delivering the gas to a bioreactor including a biological agent for promoting biomass growth. Preferably, the biological agent is cyanobacteria or algae. The method may still further include the step of harvesting at least some of the biomass from the bioreactor.

In another aspect, the fermentation process produces ethanol and stillage, and the method further includes recovering oil from the stillage. The recovered oil may be used as fuel, such as biodiesel.

In accordance with another aspect of the invention, a method of creating biomass is disclosed. The method comprises producing substantially pure CO₂ using a fermentation process, recovering the CO₂ from the fermentation process, and using the CO₂ to generate the biomass.

The method may further include the step of using the biomass in the fermentation process. Preferably, the fermentation process is a first fermentation process, and further including the step of using the biomass in a second fermentation process.

In accordance with still another aspect of the invention, a method for producing ethanol, biomass, and oxygen from ground corn is disclosed. The method comprises cooking the ground corn, fermenting the ground cooked corn to produce ethanol and CO₂, and then recovering the CO₂.

The method may further involve the step of fermenting the biomass to produce ethanol and CO₂. The step of fermenting the ground cooked corn and fermenting the biomass are preferably performed simultaneously. In any case, the step of using the CO₂ from the step of fermenting the biomass to create additional biomass and oxygen may also be performed.

In accordance with yet another aspect of the invention, a method of recycling CO₂ resulting from fermentation is disclosed. The method comprises fermenting a first biomass to produce CO₂, using the CO₂ to produce a second biomass, and fermenting the second biomass to produce CO₂. Preferably, the first biomass is corn and the second biomass comprises algae.

In accordance with still a further aspect of the invention, a method of recycling CO₂ resulting from fermentation is disclosed. The method comprises: (a) fermenting biomass to produce CO₂; (b) using the CO₂ to produce biomass; and continuously repeating steps (a) and (b).

Yet a further aspect of the invention is a system for generating biomass, comprising a fermenter for producing alcohol and CO₂, and a bioreactor including a biological agent capable of processing the CO₂ received from the fermenter to create the biomass and oxygen.

The system may further include a harvester for harvesting the biomass. Preferably, the bioreactor includes a membrane for supporting the biological agent during the processing of CO₂ to create the biomass and the harvester comprises a nozzle for spraying water to dislodge the biomass from the membrane. A delivery line may be provided for delivering the harvested biomass to the fermenter.

Preferably, the fermenter receives cooked ground corn and the alcohol is ethanol. The system may in any case include a boiler for cooking the ground corn and creating an exhaust gas, and a delivery line for delivering the exhaust gas to the bioreactor. Preferably, the fermenter is a tank and produces stillage, in which case the system comprises: (1) a separator for separating the stillage into whole stillage and thin stillage; (2) an evaporator for concentrating the thin stillage; and (3) a centrifuge (and most preferably a disk stack centrifuge) for recovering oil from the concentrated thin stillage.

In accordance with an added aspect of the invention, a system for generating biomass is disclosed. The system comprises: (1) means for producing alcohol and CO₂; and (2) means for creating the biomass and oxygen from the CO₂. Preferably, the producing means is a fermenter. It is also preferable that the creating means comprises a bioreactor including a biological agent capable of processing the CO₂ received from the fermenter to create the biomass and oxygen. the biological agent may be a cyanobacteria or an algae.

The producing means may also generate stillage. In that case, the system may further include means for recovering oil from the stillage. In one embodiment, the oil recovering means comprises: (1) means for separating the stillage into whole stillage and thin stillage; (2) means for concentrating the thin stillage; and (3) means for recovering oil from the concentrated thin stillage. Preferably, (1) the means for separating the stillage into whole stillage and thin stillage comprises a decanter; (2) the means for concentrating the thin stillage comprises an evaporator; and (3) the means for recovering oil from the concentrated thin stillage comprises a centrifuge.

### BRIEF DESCRIPTION OF THE DRAWINGS

FIG. 1 is a schematic diagram illustrating various aspects of the invention; and

FIG. 2 is a schematic diagram illustrating various aspects of the invention.

### DETAILED DESCRIPTION OF THE INVENTION

One aspect of the invention is a method and related system of forming biomass and oxygen from a byproduct resulting from a fermentation process, such as that used in the production of ethanol from corn using a dry or wet milling technique. Preferably, this byproduct is a waste gas released during the fermentation process, and includes an amount of CO₂ sufficient to sustain and encourage growth of a particular biological agent, such as cyanobacteria, to create biomass. As a result, a substantially self-contained system for the production of ethanol may result in which the gas is used for the production of biomass, which in turn can be harvested on site and used in the fermentation process for forming ethanol.

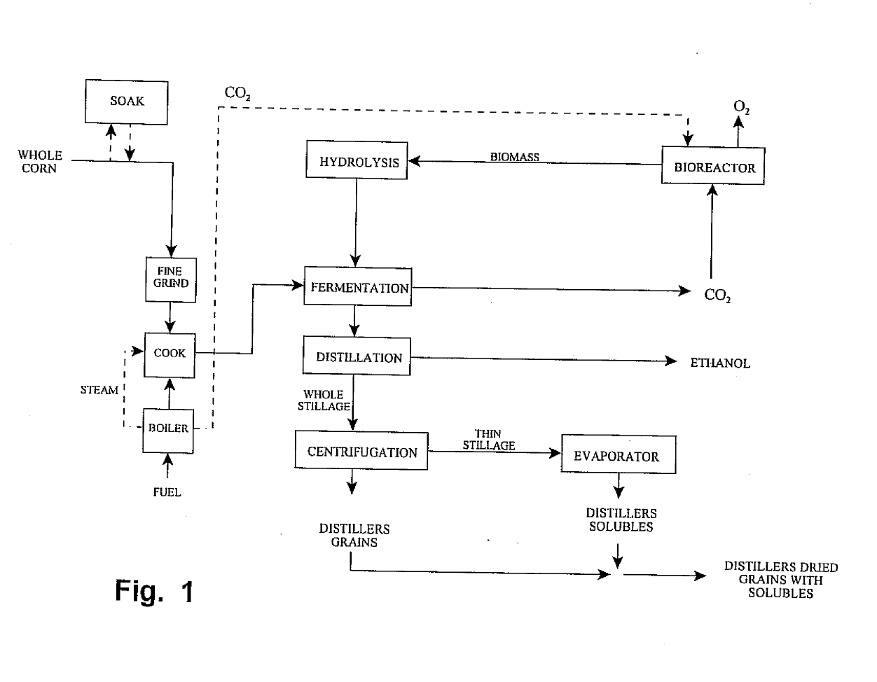

A schematic diagram illustrating one possible system and implementation of the inventive method is attached as FIG. 1. The basic dry milling process commences with finely grinding the corn and then cooking it. The cooked, ground corn is then allowed to ferment, usually in a tank with added enzymes. This fermentation of course produces the carbon dioxide (CO₂) important to one aspect of this invention. Distillation recovers the ethanol, leaving whole stillage as a byproduct.

Through various techniques using a separator, such as a centrifuge, thin stillage may be recovered from the whole stillage. This thin stillage is concentrated (such as through evaporation) to create distillers solubles. The distillers solubles is then typically combined with the distillers grains leftover from the recovery of thin stillage, and the combination dried to form distillers dried grains with solubles (DDGS).

The inventive method and system includes means for converting the CO₂ created during fermentation into more desirable byproducts, such as oxygen (O₂) that can simply be released into the atmosphere, and biomass that can be used in furtherance of the ethanol production process. The converting means is preferably a biomass generator including at least one bioreactor of the type disclosed in the above-referenced '171 patent, and preferably an array of such bioreactors. As described in detail, these bioreactors use biological agents, such as microbes (cyanobacteria) or algae, that thrive on CO₂ and generate added biomass as a result. Examples of suitable algae include those high in fat, such as botryococcus braunii, and those high in starch, such as gracilaria and chlamydomonas reinhardtii. However, any means for converting CO₂ into any type of biomass, or generating any type of biomass from CO₂, could also be used.

Once harvested, the biomass, which may include a large amount of starch in view of the upstream processing, can be used in the fermentation process for producing ethanol (either in a separate fermentation and cooking stage prior to distillation, or in the same line used to produce ethanol from the milled corn, depending on the type of enzyme action available). The byproduct of CO₂ created then goes to supply the converting means, which in turn produces more biomass. Essentially, the CO₂ is being “recycled” into products for fermentation to create more ethanol. In the illustrative example, the recycling also occurs in a most efficient fashion, since the biomass may be created at the same location where fermentation occurs, thus eliminating the need for costly, long distance transport. Also, the CO₂ (which may be substantially pure) resulting from the ethanol production may be used to feed the biomass, instead of being exhausted, undergoing costly remediation using known scrubbing techniques, or being stored indefinitely. Other uses may include any known use for CO₂, such as in the production of carbonated beverages. Having a clean source of CO₂, also allows for the use of bioreactors that are also sanitary to allow for growth of valuable algae or photosynthetic microorganisms.

The following prophetic example illustrates one possible “large scale” application of the above-described technology.

### EXAMPLE

A 50 million gallon per year ethanol plant consumes 18 million bushels of corn (at 56 lbs per bushel) that contains 695 million pounds of starch. Using the above-described dry milling process, this corn produces 336 million pounds of ethanol and 336 million pounds of CO₂. Installation of roughly 5 acres of the bioreactors of the type described in the '171 patent will convert the majority of CO₂ into oxygen and produce approximately 34 million pounds of additional starch in the form of biomass. This is enough starch to allow for an additional 5% of ethanol production (or 2.5 million gallons) and 2 million pounds of fat. The basic mass flow equation is that every three pounds of corn that enter the ethanol plant produces one pound of ethanol, one pound of distillers dried grains, and one pound of CO₂.

Advantageously, the fat, typically in the form of oil, can be recovered from the stillage. Preferably, this is done using the highly efficient and effective techniques described in U.S. patent application Ser. Nos. 11/241,231 and 11/122,859 (the disclosures of which are both incorporated herein by reference), but other processes such as solvent extraction could also be used to advantage (although at a greater cost). This oil translates to approximately 300,000 gallons of biodiesel. The net result is a total of 2.8 million gallons of renewable fuel having an annual revenue of $5.6 million, and a substantial reduction in the amount of CO₂ that would otherwise escape into the environment or require costly disposal.

As shown in FIG. 1 and noted above, the inventive method may also include a step in which hydrolysis is performed on the biomass recovered from the bioreactor and before delivery to the fermenter. As is known in the art, the hydrolysis may be performed by heating (cooking), enzyme action or the use of dilute acids. The method may also further enhance the recovery of CO₂ by delivering any CO₂-laden exhaust from any boiler (which is typically fueled by steam resulting from the combustion of gas or coal) used for cooking the ground corn to the bioreactor, as shown.

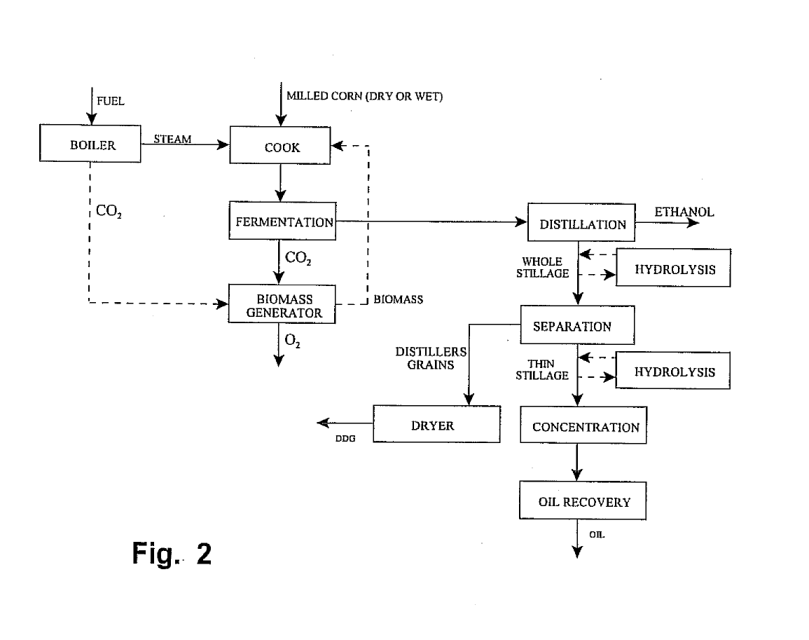

FIG. 2 is a second schematic diagram illustrating the processing of corn to produce ethanol and oil with CO₂ recycling in a slightly different way. In particular, this diagram shows that the biomass created by the biomass generator (e.g., bioreactors) can be combined with the corn and cooked using heat input (steam) from a common boiler (with the CO₂ recovered going to the biomass generator). Distillation of the fermented biomass produces ethanol and primarily whole stillage as a byproduct.

As mentioned above and in certain of the patent applications incorporated herein by reference, the whole stillage includes valuable oil that may be recovered using various techniques. Besides simply separating the whole stillage into thin stillage and distillers grains, the whole stillage may first undergo hydrolyzation in order to separate the bound oil that might not otherwise be recovered using mechanical separation techniques. This hydrolyzation may be accomplished by cooking the whole stillage under pressure to above the boiling point of water and preferably about 230°-250° F., followed by cooling and then separation to create the thin stillage with an enhanced amount of unbound oil. Alternatively, the thin stillage may be hydrolyzed after separation, but before concentration.

In either case, more oil is recovered from the resulting syrup because of hydrolyzation, which means more biofuel may be produced. The remaining products can then be dried more efficiently because of the oil removal and distillers dried grains produced. Of course, practice of the oil recovery method disclosed herein is considered entirely optional.

In the case where the biomass generated contains oil, it may proceed straight to an oil extraction step, as described above, such as through centrifugation or solvent extraction. In any case, the remaining biomass that exists after starch or oil extraction, can be used potentially as a food co-product, or feed ingredient if it contains a sufficient amount of protein.

The foregoing description provides illustration of the inventive concepts. The descriptions are not intended to be exhaustive or to limit the disclosed invention to the precise form disclosed. Modifications or variations are also possible in light of the above teachings. The embodiments described above were chosen to provide the best application to thereby enable one of ordinary skill in the art to utilize the inventions in various embodiments and with various modifications as are suited to the particular use contemplated. All such modifications and variations are within the scope of the invention.

1. A method of creating oxygen and biomass, comprising:
fermenting corn to produce ethanol and a gaseous byproduct;
recovering the gaseous byproduct; and
using the gaseous byproduct to generate the oxygen and biomass.

2. The method of claim 1, wherein the gaseous byproduct comprises CO₂.

3. The method of claim 1, further including using the biomass created to produce the gaseous byproduct.

4. The method of claim 1, further including fermenting the biomass.

5. The method of claim 1, further including extracting oil from the biomass.

6. The method of claim 1, further including hydrolyzing the biomass.

7. The method of claim 1, further including milling corn, and wherein the fermenting step uses the milled corn.

8. The method of claim 7, wherein the dry milling step includes cooking the milled corn using a boiler, and the method further includes using the boiler exhaust to generate the biomass.

9. The method of claim 1, wherein using the gaseous byproduct to generate the biomass includes delivering the gas to a bioreactor including a biological agent for promoting biomass growth.

10. The method of claim 9, wherein the biological agent comprises cyanobacteria or algae.

11. The method of claim 9, further including the step of harvesting at least some of the biomass from the bioreactor.

12. The method of claim 1, wherein the fermentation process produces ethanol and stillage, and the method further includes recovering oil from the stillage.

13. The method of claim 12, further including the step of using the oil as fuel.

14. A method of creating biomass, comprising:
producing substantially pure CO₂ using a fermentation process;
recovering the CO₂ from the fermentation process; and
using the CO₂ to generate the biomass.

15. The method of claim 14, further including the step of using the biomass in the fermentation process.

16. The method of claim 14, wherein the fermentation process is a first fermentation process, and further including the step of using the biomass in a second fermentation process.

17. A method for producing ethanol, biomass, and oxygen from ground corn, comprising:
cooking the ground corn;
fermenting the ground cooked corn to produce ethanol and CO₂; and
using the CO₂ to create biomass and oxygen.

18. The method of claim 17, further including the step of fermenting the biomass.

19. The method of claim 18, wherein the step of fermenting the ground cooked corn and fermenting the biomass are performed simultaneously.

20. The method of claim 18, further including the step of using the CO₂ from the step of fermenting the biomass to create additional biomass and oxygen.

21. The method of claim 17, further including the step of recovering oil from the biomass.

22. A method of recycling CO₂ resulting from fermentation, comprising:
fermenting a first biomass to produce CO₂;
using the CO₂ to produce a second biomass; and
fermenting the second biomass to produce CO₂.

23. The method of claim 22, further including the step of fermenting the first and second biomass together.

24. A system for generating biomass, comprising:
a fermenter for producing alcohol and CO₂; and
a bioreactor operatively connected to the fermenter and including a biological agent capable of processing the CO₂ received from the fermenter to create the biomass.

25. The system of claim 24, wherein the fermenter receives cooked ground corn and the alcohol comprises ethanol.

26. The system of claim 24, further including a boiler for cooking the ground corn and creating an exhaust gas, and a delivery line for delivering the exhaust gas to the bioreactor.

27. A system for generating biomass, comprising:
means for producing alcohol and CO₂; and
means for creating the biomass and oxygen from the CO₂.
